(19) United States
(12) Patent Application Publication
(10) Pub. No.: US 2005/0009925 A1
(43) Pub. Date: Jan. 13, 2005

(54) Use of norepinephrine reuptake inhibitors for the treatment of cognitive failure

(76) Inventors: Franklin Porter Bymaster, Brownsburg, IN (US); Donald Richard Gehlert, Indianapolis, IN (US); David McKinzie, Whitestown, IN (US); Charles Renkin Yang, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 10/496,765

(22) PCT Filed: Nov. 27, 2002
(86) PCT No.: PCT/US02/36132

Related U.S. Application Data
(60) Provisional application No. 60/339,174, filed on Dec. 11, 2001.

(51) Int. Cl. A61K 31/137

(52) U.S. Cl. 514/651

(57) ABSTRACT

Selective norepinephrine reuptake inhibitors, particularly atomoxetine, reboxetine and 2-alkylthio substituted phenoxyphenyl propylamines, are used for the treatment of cognitive failure, including cognitive failure due to dementia, delirium and schizophrenia.

The invention belongs to the fields of pharmaceutical chemistry and central nervous system medicine, and provides a method of treatment for cognitive failure.

Cognitive failure is the dysfunction or loss of cognitive functions, the processes by which knowledge is acquired, retained, and used. An estimated 2% of all Americans have some form and degree of cognitive failure, with about 15% of those over the age of 65 affected. Cognitive failure is most commonly due to delirium or dementia, but it may also occur in association with a number of other disorders.

A delerium is characterized by a disturbance of consciousness and a change in cognition that develop over a short period of time. Dementia is a chronic deterioration of intellectual function and other cognitive skills severe enough to interfere with the ability to perform activities of daily living. Although dementia may occur at any age, it primarily affects the elderly, presenting in more than 15% of persons over 65 years of age and in as many as 40% of persons over 80 years old. Dementia accounts for more than half of nursing home admissions. Dementia due to Alzheimer's disease affects four million Americans, at an annual cost of about $90 billion, including medical and nursing home care, social services, lost productivity, and early death. Alzheimer's disease accounts for more than 65% of the dementias in the elderly.

Non-Alzheimer's dementias include Lewy body dementia, vascular dementia, and Binswanger's dementia (subcortical arteriosclerotic encephalopathy). Dementia may also appear in patients with Parkinson's disease, progressive supranuclear palsy, Huntington's disease (chorea), Pick's disease, frontal lobe dementia syndromes, dementia pugilistica, normal-pressure hydrocephalus, subdural hematoma, Creutzfeldt-Jakob disease, Gerstmann-Straussler-Scheinker disease, general paresis, AIDS, and schizophrenia.

Current treatments for cognitive failure include compounds that enhance cholinergic transmission such as donepezil, rivastigmine, and tacrine. The use of these agents is limited by side effects including changes in vision or balance, diarrhea, dizziness, fainting spells or falls, increase in frequency of passing urine or incontinence, nervousness, agitation, increased confusion, skin rash or hives, slow heartbeat or palpitations, stomach pain, sweating, uncontrollable movements, unusual bleeding or bruising, red or purple spots on the skin, vomiting, and weight loss. Another therapy is the administration of the ergot hydergine. Hydergine therapy may require six months to determine whether the drug has been effective, and side effects include nausea.

Additional therapies are needed for the treatment of cognitive failure that are more efficacious and better tolerated than treatments that are currently available.

The present invention provides a method for the treatment of cognitive failure that comprises administering to a mammal in need of such treatment an effective amount of a selective norepinephrine reuptake inhibitor.

The present invention provides a method for the treatment of cognitive failure that relies on a novel mechanism of action. This method comprises treating a mammal suffering from cognitive failure with a compound that is a selective norepinephrine reuptake inhibitor. This mechanism is operative in mammals and the preferred mammal is a human.

The present invention also provides the use of a selective norepinephrine reuptake inhibitor for the preparation of a medicament useful for the treatment or prevention of cognitive failure.

The present invention provides a method for the treatment of cognitive failure. The term “cognitive failure”, as used herein, refers to the spectrum of cognitive dysfunctions ranging from mild cognitive impairment to deterioration of intellectual function and other cognitive skills severe enough to interfere with the ability to perform activities of daily living.

Many compounds, including those discussed at length below, are selective norepinephrine reuptake inhibitors, and no doubt, many more will be identified in the future. In the practice of the present invention, it is intended to include reuptake inhibitors which show 50% effective concentrations of about 1000 nM or less, in the protocol described by Wong et al., Drug Development Research, 6, 397 (1985). The norepinephrine reuptake inhibitors useful for the method of the present invention are characterized in being selective for the inhibition of neurotransmitter reuptake relative to their ability to act as direct agonists or antagonists at other receptors. It is preferred that the compounds useful for the method of the present invention are selective for the inhibition of norepinephrine reuptake relative to direct agonist or antagonist activity at other receptors by a factor of at least ten. Preferably, compounds useful for the method of the present invention are selective for the inhibition of norepinephrine reuptake relative to direct agonist or antagonist activity at other receptors by a factor of at least one hundred. Norepinephrine reuptake inhibitors useful for the method of the present invention include, but are not limited to:

Atomoxetine (formerly known as tomoxetine), (R)-(−)-N-methyl-3-(2-methylphenoxy)-3-phenylpropylamine, is usually administered as the hydrochloride salt. Atomoxetine was first disclosed in U.S. Pat. No. 4,314,081. The word “atomoxetine” will be used here to refer to any acid addition salt or the free base of the molecule. See, for example, Gehlert, et al., Neuroscience Letters, 157, 203-206 (1993), for a discussion of atomoxetine's activity as a norepinephrine reuptake inhibitor;

The compounds of formula I: 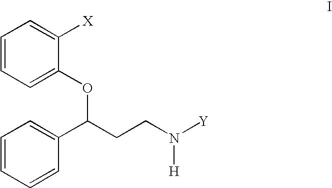
wherein X is C₁-C₄ alkylthio, and Y is C₁-C₂ alkyl or a pharmaceutically acceptable salt thereof. The compounds of formula I were described in U.S. Pat. No. 5,281,624, of Gehlert, Robertson, and Wong, and in Gehlert, et al., Life Sciences, 55(22), 1915-1920, (1995). The compounds are there taught to be inhibitors of norepinephrine reuptake in the brain. It is also explained that the compounds exist as stereoisomers, and that they accordingly include not only the racemates, but also the isolated individual isomers as well as mixtures of the individual isomers. For example, the compounds of formula I include the following exemplary species:
N-ethyl-3-phenyl-3-(2-methylthiophenoxy)propylamine benzoate;
(R)-N-methyl-3-phenyl-3-(2-propylthiophenoxy)propylamine hydrochloride;
(S)-N-ethyl-3-phenyl-3-(2-butylthiophenoxy)propylamine;
N-methyl-3-phenyl-3-(2-ethylthiophenoxy)propylamine malonate;
(S)-N-methyl-3-phenyl-3-(2-tert-butylthiophenoxy)propylamine naphthalene-2-sulfonate;
(R)-N-methyl-3-(2-methylthiophenoxy)-3-phenylpropylamine; and

Reboxetine (Edronax™), 2-[α-(2-ethoxy)phenoxybenzyl]morpholine, is usually administered as the racemate. It was first taught by U.S. Pat. No. 4,229,449, which describes its utility for the treatment of depression. Reboxetine is a selective norepinephrine reuptake inhibitor. The term “reboxetine” will be used here to refer to any acid addition salt or the free base of the molecule existing as the racemate or either enantiomer.

While all compounds exhibiting norepinephrine reuptake inhibition are useful for the methods of the present invention, certain are preferred. It is preferred that the norepinephrine reuptake inhibitor is selective for the reuptake of norepinephrine over the reuptake of other neurotransmitters. It is also preferred that the norepinephrine reuptake inhibitor does not exhibit significant direct agonist or antagonist activity at other receptors. It is especially preferred that the norepinephrine reuptake inhibitor be selected from atomoxetine, reboxetine, or (R)-N-methyl-3-(2-methylthiophenoxy)-3-phenylpropylamine. The use of atomoxetine hydrochloride for the methods of the present invention is the most preferred embodiment of the present invention.

A further embodiment of this invention comprises the administration of a composition that exhibits selective norepinephrine reuptake inhibitor activity. The composition may be composed of one or more agents that, individually or together, are selective inhibitors of norepinephrine reuptake.

The dosages of the drugs used in the present invention must, in the final analysis, be set by the physician in charge of the case using knowledge of the drugs, the properties of the drugs in combination as determined in clinical trials, and the characteristics of the patient including diseases other than that for which the physician is treating the patient. General outlines of the dosages, and some preferred dosages, can and will be provided here.

Atomoxetine: In adults and older adolescents: from about 5 mg/day to about 200 mg/day; preferably in the range from about 60 to about 150 mg/day; more preferably from about 60 to about 130 mg/day; and still more preferably from about 50 to about 120 mg/day; in children and younger adolescents: from about 0.2 to about 3.0 mg/kg/day; preferably in the range from about 0.5 to about 1.8 mg/kg/day;

Compounds of formula I: from about 0.01 mg/kg to about 20 mg/kg; preferred daily doses will be from about 0.05 mg/kg to 10 mg/kg; ideally from about 0.1 mg/kg to about 5 mg/kg;

Reboxetine: from about 1 to about 30 mg, once to four times/day; preferred, from about 5 to about 30 mg once/day.

Cognitive failure presents in patients suffering from a number of other disorders. The present invention includes the use of a norepinephrine reuptake inhibitor to treat cognitive failure presenting alone or where cognitive failure is associated with another disorder. Schizophrenic patients, for example, commonly exhibit symptoms that include cognitive failure. An embodiment of the present invention, therefore, is the use of a norepinephrine reuptake inhibitor to treat cognitive failure associated with schizophrenia. Patients suffering from schizophrenia also frequently exhibit negative symptoms such as flat affect, asociality, anergia, avolition, and anhedonia. A further embodiment of the present invention is the use of a norepinephrine reuptake inhibitor to treat the negative symptoms of schizophrenia.

The invention further provides a method for treating a patient suffering from or susceptible to psychosis, comprising administering to said patient an effective amount of a first component which is an antipsychotic, in combination with an effective amount of a second component which is a norepinephrine reuptake inhibitor. The invention also provides a pharmaceutical composition that comprises a first component that is an antipsychotic, and a second component that is a norepinephrine reuptake inhibitor.

In the general expressions of this aspect of the present invention, the first component is a compound that acts as an antipsychotic. The antipsychotic may be either a typical antipsychotic, such as haloperidol, or an atypical antipsychotic. The essential feature of an atypical antipsychotic is less acute extrapyramidal symptoms, especially dystonias, associated with therapy as compared to a typical antipsychotic such as haloperidol. Clozapine, the prototypical atypical antipsychotic, differs from the typical antipsychotics with the following characteristics: (1) greater efficacy in the treatment of overall psychopathology in patients with schizophrenia nonresponsive to typical antipsychotics; (2) greater efficacy in the treatment of negative symptoms of schizophrenia; and (3) less frequent and quantitatively smaller increases in serum prolactin concentrations associated with therapy (Beasley, et al., Neuropsychopharma-cology, 14(2), 111-123, (1996)). Although both typical and atypical antipsychotics are useful for these methods and formulations of the present invention, it is preferred that the first component compound is an atypical antipsychotic.

Typical antipsychotics include, but are not limited to:

Chlorpromazine, 2-chloro-10-(3-dimethylaminopropyl)phenothiazine, is described in U.S. Pat. No. 2,645,640. Its pharmacology has been reviewed (Crismon, Psychopharmacol. Bul., 4, 151 (October 1967);

Droperidol, 1-(1-[3-(p-fluorobenzoyl)propyl]-1,2,3,6-tetrahydro-4-pyridyl)-2-benzimidazolinone, is described in U.S. Pat. No. 3,141,823;

Haloperidol, 4-[4-(4-chlorophenyl)-4-hydroxy-1-piperidinyl]-1-(4-fluorophenyl)-1-butanone, is described in U.S. Pat. No. 3,438,991. Its therapeutic efficacy in psychosis has been reported (Beresford and Ward, Drugs, 33, 31-49 (1987);

Thioridazine, 1-hydroxy-10-[2-(1-methyl-2-pyridinyl)ethyl]-2-(methylthio)phenothiazine hydrochloride, was described by Bourquin, et al. (Helv. Chim. Acta, 41, 1072 (1958)). Its use as an antipsychotic has been reported (Axelsson, et al., Curr. Ther. Res., 21, 587 (1977)); and

Trifluoperazine, 10-[3-(4-methyl-1-piperazinyl)propyl]-2-trifluoromethylphenthiazine hydrochloride, is described in U.S. Pat. No. 2,921,069.

Atypical antipsychotics include, but are not limited to: Olanzapine, 2-methyl-4-(4-methyl-1-piperazinyl)-10H-thieno[2,3-b][1,5]benzodiazepine, is a known compound and is described in U.S. Pat. No. 5,229,382 as being useful for the treatment of schizophrenia, schizophreniform disorder, acute mania, mild anxiety states, and psychosis;

Clozapine, 8-chloro-11-(4-methyl-1-piperazinyl)-5H-dibenzo[b,e][1,4]diazepine, is described in U.S. Pat. No. 3,539,573. Clinical efficacy in the treatment of schizophrenia is described (Hanes, et al., Psychopharmacol. Bull., 24, 62 (1988));

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663;

Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945;

Quetiapine, 5-[2-(4-dibenzo[b,f][1,4]thiazepin-11-yl-1-piperazinyl)ethoxy]ethanol, and its activity in assays which demonstrate utility in the treatment of schizophrenia are described in U.S. Pat. No. 4,879,288. Quetiapine is typically administered as its (E)-2-butenedioate (2:1) salt; and

Ziprasidone, 5-[2-[4-(1,2-benzoisothiazol-3-yl)-1-piperazinyl]ethyl]-6-chloro-1,3-dihydro-2H-indol-2-one, is typically administered as the hydrochloride monohydrate.

The compound is described in U.S. Pat. Nos. 4,831,031 and 5,312,925. Its activity in assays which demonstrate utility in the treatment of schizophrenia are described in U.S. Pat. No. 4,831,031.

Similarly, when this aspect of the invention is regarded in its broadest sense, the second component compound is a compound that functions as a norepinephrine reuptake inhibitor as described above.

It will be understood that while the use of a single antipsychotic as a first component compound is preferred, combinations of two or more antipsychotics may be used as a first component if necessary or desired. Similarly, while the use of a single norepinephrine reuptake inhibitor as a second component compound is preferred, combinations of two or more norepinephrine reuptake inhibitors may be used as a second component if necessary or desired.

While all combinations of first and second component compounds are useful and valuable, certain combinations are particularly valued and are preferred, as follows:
olanzapine/atomoxetine
olanzapine/reboxetine
olanzapine/(R)-N-methyl-3-(2-methylthiophenoxy)-3-phenylpropylamine
clozapine/atomoxetine
risperidone/atomoxetine
sertindole/atomoxetine
quetiapine/atomoxetine
ziprasidone/atomoxetine

In general, combinations and methods of treatment using olanzapine as the first component are preferred. Furthermore, combinations and methods of treatment using atomoxetine as the second component are preferred. Especially preferred are combinations and methods of treatment using olanzapine as the first component and atomoxetine as the second component. It is especially preferred that when the first component is olanzapine, it will be the Form II olanzapine as described in U.S. Pat. No. 5,736,541.

It is further preferred that the Form II olanzapine polymorph will be administered as the substantially pure Form II olanzapine polymorph. As used herein “substantially pure” refers to Form II associated with less than about 5% Form I, preferably less than about 2% Form I, and more preferably less than about 1% Form I. Further, “substantially pure” Form II will contain less than about 0.5% related substances, wherein “related substances” refers to undesired chemical impurities or residual solvent or water. In particular, “substantially pure” Form II should contain less than about 0.05% content of acetonitrile, more preferably, less than about 0.005% content of acetonitrile. Additionally, the polymorph of the invention should contain less than 0.5% of associated water.

Although Form II olanzapine is preferred it will be understood that as used herein, the term “olanzapine” embraces all solvate and polymorphic forms unless specifically indicated.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them. Especially preferred pharmaceutically acceptable salts are those formed with hydrochloric acid.

The dosages of the first component drugs used in this aspect of the present invention must, in the final analysis, be set by the physician in charge of the case, using knowledge of the drugs, the properties of the drugs in combination as determined in clinical trials, and the characteristics of the patient, including diseases other than that for which the physician is treating the patient. General outlines of the dosages, and some preferred dosages, can and will be provided here. Dosage guidelines for some of the drugs will first be given separately; in order to create a guideline for any desired combination, one would choose the guidelines for each of the component drugs.
Chlorpromazine: from about 25-75 mg daily to about 75-150 mg daily;
Droperidol: about 5 mg by injection;
Haloperidol: from about 1-15 mg/day to about 100 mg/day administered orally or by injection;
Thioridazine: about 75-150 mg daily;
Trifluoperazine: from about 4-10 mg/day to about 15-20 mg/day;
Olanzapine: from about 0.25 to 50 mg, once/day; preferred, from 1 to 30 mg, once/day; and most preferably 1 to 25 mg once/day;
Clozapine: from about 12.5 to 900 mg daily; preferred, from about 150 to 450 mg daily;
Risperidone: from about 0.25 to 16 mg daily; preferred from about 2-8 mg daily;
Sertindole: from about 0.0001 to 1.0 mg/kg daily;
Quetiapine: from about 1.0 to 40 mg/kg given once daily or in divided doses;
Ziprasidone: from about 5 to 500 mg daily; preferred from about 50 to 100 mg daily.

In more general terms, one would create a combination of the present invention by choosing a dosage of first and second component compounds according to the spirit of the above guideline.

The adjunctive therapy aspect of the present invention is carried out by administering a first component together with the second component in any manner that provides effective levels of the compounds in the body at the same time. Oral administration of the adjunctive combination is preferred. Both components may be administered together, in a single dosage form, or may be administered separately. However, oral administration is not the only route or even the only preferred route. For example, transdermal administration may be very desirable for patients who are forgetful or petulant about taking oral medicine. Administration by the percutaneous, intravenous, intramuscular, intranasal or intrarectal route may be prudent in particular circumstances. The route of administration may be varied in any way, limited by the physical properties of the drugs, the convenience of the patient and the caregiver, and other relevant circumstances (Remington's Pharmaceutical Sciences, 18th Edition, Mack Publishing Co. (1990)).

The adjunctive combination may be administered as a single pharmaceutical composition, and so pharmaceutical compositions incorporating both compounds are important embodiments of the present invention. Such compositions may take any physical form that is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The pharmaceutical compositions are prepared in a manner well known in the pharmaceutical art. The carrier or excipient may be a solid, semi-solid, or liquid material that can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solutions, suspensions, or the like.

The compounds useful for the method of the present invention may be administered orally, for example, with an inert diluent or capsules or compressed into tablets. For the purpose of oral therapeutic administration, the compounds may be incorporated with excipients and used in the form of tablets, troches, capsules, elixirs, suspensions, syrups, wafers, chewing gums and the like. These preparations should contain at least 4% of the compound of the present invention, the active ingredient, but may be varied depending upon the particular form and may conveniently be between 4% to about 70% of the weight of the unit. The amount of the compound present in compositions is such that a suitable dosage will be obtained. Preferred compositions and preparations useful for the methods of the present invention may be determined by a person skilled in the art.

The tablets, pills, capsules, troches, and the like may also contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, as coatings. Thus, tablets or pills may be coated with sugar, shellac, or other coating agents. A syrup may contain, in addition to the present compounds, sucrose as a sweetening agent and certain preservatives, dyes and colorings and flavors. Materials used in preparing these various compositions should be pharmaceutically pure and non-toxic in the amounts used.

A formulation useful for the administration of R(−)-N-methyl 3-((2-methylphenyl)oxy)-3-phenyl-1-aminopropane hydrochloride (atomoxetine) comprises a dry mixture of R-(−)-N-methyl 3-((2-methylphenyl)oxy)-3-phenyl-1-aminopropane hydrochloride with a diluent and lubricant. A starch, such as pregelatinized corn starch, is a suitable diluent and a silicone oil, such as dimethicone, a suitable lubricant for use in hard gelatin capsules. Suitable formulations are prepared containing about 0.4 to 26% R-(−)-N-methyl 3-((2-methylphen-yl)oxy)-3-phenyl-1-aminopropane hydrochloride, about 73 to 99% starch, and about 0.2 to 1.0% silicone oil. The following tables illustrate particularly preferred atomoxetine formulations:

| | | | | | | | |
|---|---|---|---|---|---|---|---|
| 2.5 mg | 5 mg | 10 mg | 18 mg | 20 mg | 25 mg | 40 mg | 60 mg |

Ingredient (%)

| | | | | | | | | |
|---|---|---|---|---|---|---|---|---|
| R-(−)-N-methyl 3- | 1.24 | 2.48 | 4.97 | 8.94 | 9.93 | 12.42 | 19.87 | 22.12 |
| ((2-methylphenyl) | | | | | | | | |
| oxy)-3- | | | | | | | | |
| phenyl-1- | | | | | | | | |
| aminopropane | | | | | | | | |
| hydrochloride | | | | | | | | |
| Dimethicone | 0.5 | 0.5 | 0.5 | 0.5 | 0.5 | 0.5 | 0.5 | 0.5 |
| Pregelatinized | 98.26 | 97.02 | 94.53 | 90.56 | 89.57 | 87.08 | 79.63 | 77.38 |
| Starch | | | | | | | | |
| Ingredient | | | | | | | | |
| (mg/capsule) | | | | | | | | |
| R-(−)-N-methyl 3- | 2.86 | 5.71 | 11.43 | 20.57 | 22.85 | 28.57 | 45.71 | 68.56 |
| ((2-methylphenyl) | | | | | | | | |
| oxy)-3- | | | | | | | | |
| phenyl-1- | | | | | | | | |
| aminopropane | | | | | | | | |
| hydrochloride | | | | | | | | |
| Dimethicone | 1.15 | 1.15 | 1.15 | 1.15 | 1.15 | 1.15 | 1.15 | 1.55 |
| Pregelatinized | 225.99 | 223.14 | 217.42 | 208.28 | 206.00 | 200.28 | 183.14 | 239.89 |
| Starch | | | | | | | | |
| Capsule Fill Weight | 230 | 230 | 230 | 230 | 230 | 230 | 230 | 310 |
| (mg) | | | | | | | | |
| Capsule Size | 3 | 3 | 3 | 3 | 3 | 3 | 3 | 2 |

For the purpose of parenteral therapeutic administration, the compounds of the present invention may be incorporated into a solution or suspension. These preparations typically contain at least 0.1% of a compound of the invention, but may be varied to be between 0.1 and about 90% of the weight thereof. The amount of the compound of formula I present in such compositions is such that a suitable dosage will be obtained. The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Preferred compositions and preparations may be determined by one skilled in the art.

Inhibition or Norepinephrine Reuptake

The ability of compounds to inhibit the reuptake of norepinephrine may be measured by the general procedure of Wong, et al., supra.

Male Sprague-Dawley rats weighing 150-250 gm are decapitated and brains are immediately removed. Cerebral cortices are homogenized in 9 volumes of a medium containing 0.32 M sucrose and 10 mM glucose. Crude synaptosomal preparations are isolated after differential centrifugation at 1000×g for 10 minutes and 17,000×g for 28 minutes. The final pellets are suspended in the same medium and kept in ice until use within the same day.

Synaptosomal uptake of ³H-norepinephrine is determined as follows. Cortical synaptosomes (equvalent to 1 mg of protein) are incubated at 37° C. for 5 minutes in 1 mL Krebs-bicarbonate medium containing also 10 mM glucose, 0.1 mM iproniazide, 1 mM ascorbic acid, 0.17 mM EDTA and 50 nM ³H-norepinephrine. The reaction mixture is immediately diluted with 2 mL of ice-chilled Krebs-bicarbonate buffer and filtered under vacuum with a cell harvester (Brandel, Gaithersburg, Md.). Filters are rinsed twice with approximately 5 mL of ice-chilled 0.9% saline and the uptake of ³H-norepinephrine assessed by liquid scintillation counting. Accumulation of ³H-norepinephrine at 4° C. is considered to be background and is subtracted from all measurements. The concentration of the test compound required to inhibit 50% of the ³H-norepinephrine accumulation (IC₅₀ values) are determined by linear regression analysis.

The present invention provides a method for the treatment of cognitive failure. Cognitive failure may present in patients suffering from a number of disorders. The methods of the present invention are useful for the treatment of cognitive failure associated with disorders classified in the Diagnostic and Statistical Manual of Mental Disorders, 4th Version, published by the American Psychiatric Association (DSM-IV). The DSM code numbers are supplied below for the convenience of the reader.

| | |
|---|---|
| Delirium Due to a General Medical Condition | 293.0 |
| Delirium Not Otherwise Specified | 780.09 |
| Dementia of the Alzheimer's Type | |
| Early Onset with Delirium | 290.11 |
| Early Onset with Delusions | 290.12 |
| Early Onset Uncomplicated | 290.10 |
| Late Onset with Delirium | 290.3 |
| Late Onset with Delusions | 290.20 |
| Late Onset Uncomplicated | 290.0 |
| Vascular Dementia | |
| With Delirium | 290.41 |
| With Delusions | 290.42 |
| Uncomplicated | 290.40 |
| Dementia Due to HIV Disease | 294.1 |
| Dementia Due to Head Trauma | 294.1 |
| Dementia Due to Parkinson's Disease | 294.1 |
| Dementia Due to Huntington's Disease | 294.1 |
| Dementia Due to Pick's Disease | 290.10 |
| Dementia Due to Creutzfeldt-Jakob Disease | 290.10 |
| Dementia Due to Other General Medical Conditions | 294.1 |
| Dementia Not Otherwise Specified | 294.8 |
| Amnestic Disorder Due to a General Medical | 294.0 |
| Condition | |
| Amnestic Disorder Not Otherwise Specified | 294.8 |
| Cognitive Disorder Not Otherwise Specified | 294.9 |
| Paranoid Type Schizophrenia | 295.30 |
| Disorganized Type Schizophrenia | 295.10 |
| Catatonic Type Schizophrenia | 295.20 |
| Undifferentiated Type Schizophrenia | 295.90 |
| Residual Type Schizophrenia | 295.60 |
| Schizophreniform Disorder | 295.40 |
| Schizoaffective Disorder | 295.70 |

The skilled artisan will appreciate that the disorders listed above are illustrative of those indications where cognitive failure may appear, and is not intended to limit the scope of the present invention in any way.

Psychotic conditions to be treated by the adjunctive therapy aspect of the present invention include schizophrenia, schizophreniform diseases, acute mania, and schizoaffective disorders. The titles given these conditions represent multiple disease states. The following list illustrates a number of these disease states, many of which are classified in the Diagnostic and Statistical Manual of Mental Disorders, 4th Edition, published by the American Psychiatric Association (DSM). The DSM code numbers for these disease states are supplied below, when available, for the convenience of the reader.

| | |
|---|---|
| Paranoid Type Schizophrenia | 295.30 |
| Disorganized Type Schizophrenia | 295.10 |
| Catatonic Type Schizophrenia | 295.20 |
| Undifferentiated Type Schizophrenia | 295.90 |
| Residual Type Schizophrenia | 295.60 |
| Schizophreniform Disorder | 295.40 |
| Schizoaffective Disorder | 295.70 |

The present invention is also useful for the treatment of cognitive failure related to the onset of menopause.

The method of the present invention is effective in the treatment of patients who are children, adolescents or adults, and there is no significant difference in the symptoms or the details of the manner of treatment among patients of different ages. In general terms, however, for purposes of the present invention, a child is considered to be a patient below the age of puberty, an adolescent is considered to be a patient from the age of puberty up to about 18 years of age, and an adult is considered to be a patient of 18 years or older.

### EXAMPLE 1

The immediate-early gene, c-fos, and its protein products have been increasingly utilized as markers for neuronal activation (Dragunow and Faull, J. Neurosci. Methods, 29, 261-265 (1989); Morgan and Curran, Prog. In Brain Res., 86, 287-294 (1990); Robertson, et al., J. Pharmacol. Exp. Ther., 271, 1058-1066 (1994)). C-fos activation is measured as illustrated below for atomoxetine.

Two hours after administration of atomoxetine (3 mg/kg, i.p.), the rats were deeply anesthetized with sodium pentobarbital (60 mg/kg, i.p.) and transcardially perfused with 100 ml of phosphate buffered saline (PBS) followed by 100 ml of 4% paraformaldehyde in PBS. The brain was rapidly removed, postfixed for 90 min in 4% paraformaldehyde and then was transferred to 30% sucrose at 40 C until saturated. After quick freezing, serial 30 μm sections were cut and placed in PBS until processed for immunohistochemistry. In brief, sections were incubated in PBS containing blocking serum and 0.5% Triton-X 100 for 1 hour. Sections were then incubated with anti-Fos antibody (Santa Cruz Biotechnology, Inc.) at 4° C. overnight. Visualization of the Fos-like immunoreactivity was performed with a Vectastain ABC Elite Kit (Vector Labs, Burlingame, Calif.) using the standard protocol supplied with the kit. Nickel-intensified diaminobenzidine (DAB) was used as the chromagen to yield a gray-black precipitation product. Following visualization of the Fos immunoreactivity, the sections were mounted on gelatin-coated glass slides and allowed to dry. The sections were then dehydrated and cover slipped. Fos expressing cells were quantitated using the MCID M2 imaging system (Imaging Research, St. Catherines, Ontario).

Surprisingly, atomoxetine increased the expression of c-fos only in the cortical areas as demonstrated by the data in the following table.

| | Prefrontal | Nucleus | |
|---|---|---|---|
| Treatment | Cortex** | Accumbens** | Striatum** |
| Vehicle | 80 ± 28 | 129 ± 33 | 118 ± 26 |
| Atomoxetine | 296 ± 26* | 152 ± 44 | 102 ± 35 |

*= p < 0.001
**Fos positive cells/mm²

1-15. (canceled).

16. A method of treating cognitive failure, comprising administering to a human patient in need thereof an effective amount of a selective norepinephrine reuptake inhibitor selected from the group consisting of atomoxetine and a compound of formula I: 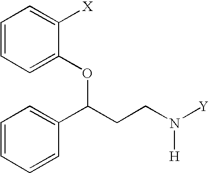
wherein X is C₁-C₄ alkylthio, and Y is C₁-C₂ alkyl,
or a pharmaceutically acceptable salt thereof.

17. The method of claim 16, wherein said selective norepinephrine reuptake inhibitor is atomoxetine.

18. The method of claim 17, wherein said atomoxetine is in the form of a hydrochloride salt.

19. The method of claim 16, wherein said cognitive failure is associated with a dementia.

20. The method of claim 19, wherein said dementia is selected from the group consisting of dementia of the Alzheimer's type, vascular dementia, dementia due to head trauma, dementia due to Parkinson's Disease, dementia due to other general medical conditions, and dementia not otherwise specified.

21. The method of claim 20, wherein said human patient is an older adolescent or an adult, and said atomoxetine is administered in the form of a hydrochloride salt in a dose of from about 5 mg/day to about 200 mg/day.

22. The method of claim 20, wherein said human patient is a child or a younger adolescent, and said atomoxetine is administered in the form of a hydrochloride salt in a dose of from about 0.2 mg/kg/day to about 3.0 mg/kg/day.

23. The method of claim 16, wherein said cognitive failure is associated with a delirium.

24. The method of claim 23, wherein said delirium is selected from the group consisting of a delirium due to a general medical condition and a delirium not otherwise specified.

25. The method of claim 24, wherein said human patient is an older adolescent or an adult, and said atomoxetine is administered in the form of a hydrochloride salt in a dose of from about 5 mg/day to about 200 mg/day.

26. The method of claim 24, wherein said human patient is a child or a younger adolescent, and said atomoxetine is administered in the form of a hydrochloride salt in a dose of from about 0.2 mg/kg/day to about 3.0 mg/kg/day.

27. The method of claim 16, wherein said cognitive failure is associated with schizophrenia.

28. The method of claim 27, wherein said human patient is an older adolescent or an adult, and said atomoxetine is administered in the form of hydrochloride salt in a dose of from about 5 mg/day to about 200 mg/day.

29. The method of claim 27, wherein said human patient is a child or a younger adolescent, and said atomoxetine is administered in the form of a hydrochloride salt in a dose of from about 0.2 mg/kg/day to about 3.0 mg/kg/day.

30. A method of treating negative symptoms of schizophrenia, comprising administering to a human patient in need thereof an effective amount of a selective norepinephrine reuptake inhibitor selected from the group consisting of atomoxetine, reboxetine, and a compound of formula I: 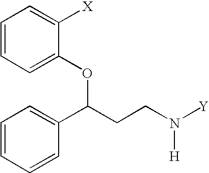
wherein X is C₁-C₄ alkylthio, and Y is C₁-C₂ alkyl,
or a pharmaceutically acceptable salt thereof.

31. The method of claim 30, wherein said human patient is an older adolescent or an adult, and said atomoxetine is administered in the form of hydrochloride salt in a dose of from about 5 mg/day to about 200 mg/day.

32. The method of claim 30, wherein said human patient is a child or a younger adolescent, and said atomoxetine is administered in the form of a hydrochloride salt in a dose of from about 0.2 mg/kg/day to about 3.0 mg/kg/day.

33. A method of treating psychosis, comprising administering to a human patient in need thereof an effective amount of a selective norepinephrine reuptake inhibitor selected from the group consisting of atomoxetine, reboxetine, and a compound of formula I: 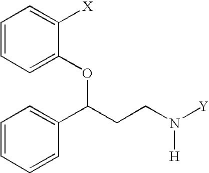
wherein X is C₁-C₄ alkylthio, and Y is C₁-C₂ alkyl,
or a pharmaceutically acceptable salt thereof, in combination with an effective amount of an antipsychotic.

34. The method of claim 33, wherein said psychosis is selected from the group consisting of schizophrenia, a schizophreniform disease, acute mania, and a schizoaffective disorder.

35. The method of claim 34, wherein said human patient is an older adolescent or an adult, and said atomoxetine is administered in the form of hydrochloride salt in a dose of from about 5 mg/day to about 200 mg/day.

36. The method of claim 34, wherein said human patient is a child or a younger adolescent, and said atomoxetine is administered in the form of a hydrochloride salt in a dose of from about 0.2 mg/kg/day to about 3.0 mg/kg/day.
